(19) United States
(12) Patent Application Publication
(10) Pub. No.: US 2008/0171029 A1
(43) Pub. Date: Jul. 17, 2008

(54) Beauty mask

(76) Inventor: Jyh-Hau Lee, Taipei City (TW)

Correspondence Address: PRO-TECHTOR INTERNATIONAL SERVICES, 20775 NORADA CT., SARATOGA, CA, 95070, US

(21) Appl. No.: 11/652,883

(22) Filed: Jan. 12, 2007

(51) Int. Cl. A61K 9/00; A61Q 19/00; A42B 1/00; A61K 38/43

(52) U.S. Cl. 424/94.1; 424/402; 2/206

(57) ABSTRACT

A beauty mask uses natural material, like coconut milk, royal jelly, vegetable enzymes, aloe and loofah, for achieving a whitening, anti-wrinkle and moisturizing effect without further additives, thereby avoiding skin injuries, and which can be stored for a long time; furthermore a method for packing and storing beauty masks, by inserting separating foils between beauty masks and comprising steps of packing, sealing and freezing, prevents beauty masks from sticking together, so that beauty masks are storable for a long time and easily dispensable.

### FIELD OF THE INVENTION

The present invention relates to a beauty mask, particularly to a beauty mask which uses natural material, like coconut milk, royal jelly, vegetable enzymes, aloe and loofah, for achieving a whitening, anti-wrinkle and moisturizing effect without further additives, thereby avoiding skin injuries, and which can be stored for a long time; furthermore to a method for packing and storing beauty masks which, by inserting separating foils between beauty masks and in steps of packing, sealing and freezing prevents beauty masks from gluing together, so that beauty masks are storable for a long time and easily dispensable.

### BACKGROUND OF THE INVENTION

Various face masks are available on the market, which are distinguished by used materials and beauty agents for tightening, whitening and moisturizing the skin as well as for reducing wrinkles.

To achieve desired effects, various organic agents are used in face masks, glycerol, alcohol, synthesized scent, emulsifying agent, essence, antiseptic agents and antioxidants. Most of these agents are synthesized rather than natural, and repeated exposure damages facial skin.

Hence agents in currently available beauty masks are harmful for skin.

Furthermore, for storing beauty masks, antiseptic agents and antioxidants are added, which, due to chemical instablility at freezing temperatures, may not be frozen.

Thus harmful antiseptic agents and antioxidants are added for storage of beauty masks, which turn out to be harmful for skin.

Moreover, for a fresh feeling of treated faces and since masks due to chemical instability of agents may not be frozen, alcohol and mint are added. These materials, however, in some cases are subject to allergies and are even banned from use in some countries.

Therefore, adding alcohol and mint to beauty masks further harms facial skin.

From what has been said above, material and agents of conventional beauty masks as well as methods of storing thereof poses a great problem, so that there is e need for a beauty mask which is effective while having no chemical additives.

### SUMMARY OF THE INVENTION

It is an object of the present invention to provide a beauty mask which is effective without being harmful to facial skin and which can be stored for extended periods. For achieving this object, the beauty mask of the present invention uses natural material, like coconut milk, royal jelly, vegetable enzymes, e.g., papayase, aloe and loofah, for achieving a whitening, anti-wrinkle and moisturizing effect without further additives, thereby avoiding skin injuries, and which can be stored for a long time. Furthermore, the present invention is directly frozen, allowing for storage over extended periods, without a need for adding antiseptic agents and antioxidants.

Another object of the present invention is to provide a method for storing of beauty masks over extended periods while maintaining freshness thereof. For achieving this object, the method of the present invention comprises the steps of (1) manufacturing a separating foil; (2) soaking a beauty mask in a liquid beauty agent; (3) fastening the beauty mask on the separating foil; (4) packing the beauty mask and the separating foil in a bag; (5) sealing the bag; and (6) freezing the bag for storage. Freezing temperatures subsequently maintain effectiveness and freshness of the beauty mask.

A further object of the present invention is to provide a beauty mask storing and dispensing device for storing of beauty masks at freezing temperatures and dispensing thereof. For achieving this object, the beauty mask storing and dispensing device of the present invention comprises bags, beauty masks and separating foils, which have openings at fixed positions into which jutting out parts of beauty masks that have been laid on separating foils are tucked. Thereby adjacent beauty masks are separated and will not glue together after freezing thereof, allowing for easy dispensing thereof.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 is a flow chart of the method for storing of beauty masks of the present invention.

FIG. 2 is a front view of the separating foil of the present invention.

FIG. 3 is a front view of the beauty mask of the present invention when fastened to the separating foil.

FIG. 4 is a rear view of the beauty mask of the present invention when fastened to the separating foil.

FIG. 5 is a schematic illustration of packing of beauty masks on separating foils of the present invention.

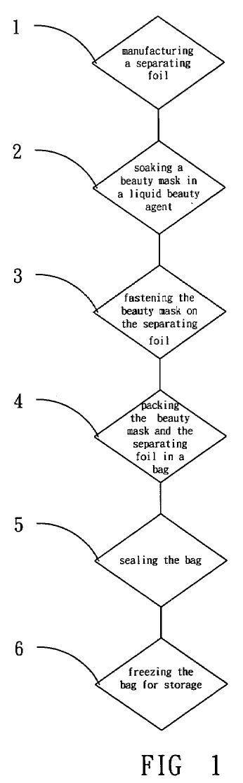

### DESCRIPTION OF THE PREFERRED EMBODIMENT

The present invention is a beauty mask which uses natural material, like coconut milk, royal jelly, vegetable enzymes, aloe and loofah, placed on a mask body made of natural material, so that facial skin is not harmed, while a whitening, anti-wrinkle and moisturizing effect is achieved without further additives. Furthermore, the present invention is directly frozen, without adding antiseptic agents and antioxidants, allowing for storage over extended periods. In the following, a detailed description is given.

Coconut milk is used to a fraction of 70%-99% and is the cleanest natural juice found in the world, since palm trees filter out germs in several layers. Coconut milk contains vitamin C, fat, proteins, fructose and glucose and is recognized as a mild, germ-free, fever-preventing liquid which is applicable to human skin and easily absorbed there, without invoking allergies. The ingredients of coconut milk have a whitening, anti-wrinkle and moisturizing effect. Soap made of palm oil is environmentally friendly and can be washed off by water, while a pleasant natural scent remains, so that no essences need to be added.

Royal jelly is used to a fraction of 0.1%-10% and has in Japan used as a beauty agent for a long time. Components of royal jelly are active proteins and 10-hydroxy-decenoic acid (10-HDA), which is naturally effective against germs, several vitamins, including A, C, E, B1, B2, B6, B12, which are beneficial to the skin, and various organic acids, including pantothenic acid, eolic acid, Inoshol and acetyl cholne. In Japan, royal jelly has a reputation of being valuable and a symbol of beauty, providing the skin with shine and brilliance.

Vegetable enzymes are used to a fraction of 0.1%-5% and soften horny skin by furthering metabolism with the help of papayase and other enzymes. Papayase quickly enters the skin and, in both an acid and alkaline environment, dissolves hair follicles, eliminates dirt and reduces pimples.

Aloe is used to a fraction of 0.1%-5% and strengthens body immunity and helps healing of wounds. The present invention uses aloe arborescens, alow vera, aloe ferox and alow perryi.

Loofah is used to a fraction of 0.1%-5% and helps to reduce swellings and pimples.

The mask of the present invention has a mask body made of nonwoven fabric with high absorption for accommodating beauty agents and having properties that do not change after prolonged exposure to freezing temperatures.

By employing coconut milk, royal jelly, vegetable enzymes, aloe and loofah, the mask of the present invention contains no substances that are harmful to human skin. The mask of the present invention is directly frozen, without adding antiseptic agents and antioxidants, and then stored at a temperature of approximately −20° C., allowing for unlimited storage. Effects for beauty are: moisturizing and reducing wrinkles by coconut milk and aloe, beautifying and preventing infection by royal jelly, refining and whitening by vegetable enzymes, which all in a short time inter depths of the skin, dissolving hair follicles and fat and giving the skin a healthy, shining appearance.

The method for manufacturing of beauty masks of the present invention comprises the steps of:

(1) mixing coconut milk to a fraction of 70%-99%, royal jelly to a fraction of 0.1%-10%, vegetable enzymes to a fraction of 0.1%-5%, aloe to a fraction of 0.1%-5%, and loofah to a fraction of 0.1%-5%, forming a liquid beauty agent, keeping the beauty agent at a temperature of 5° C. to 10° C.;
(2) filtering the beauty agent at a temperature of 5° C. to 10° C.;
(3) soaking a beauty mask body in the beauty agent, so that a beauty mask is generated.

For manufacturing beauty masks according to the present invention, it has to be considered that coconut milk is a seasonal product. For avoiding infection with germs, coconut milk used outside harvesting seasons preferably is after harvesting kept at a temperature of approximately −40° C. Royal jelly is preferably cleaned of herbicides and pesticides before usage. Papayase is preferably prepared by collecting and exposing to ultraviolet light juice of the papaya fruit, so as to kill germs. Aloe is peeled, mixed with coconut milk, filtered and exposed to ultraviolet light. Loofah is preferably exposed to ultraviolet light, as well.

Preparing the coconut milk comprises the steps of:

(1) mechanically washing coconuts;
(2) quick drying;
(3) drilling through husks of coconuts and extracting coconut milk; and
(4) storing coconut milk at freezing temperature.

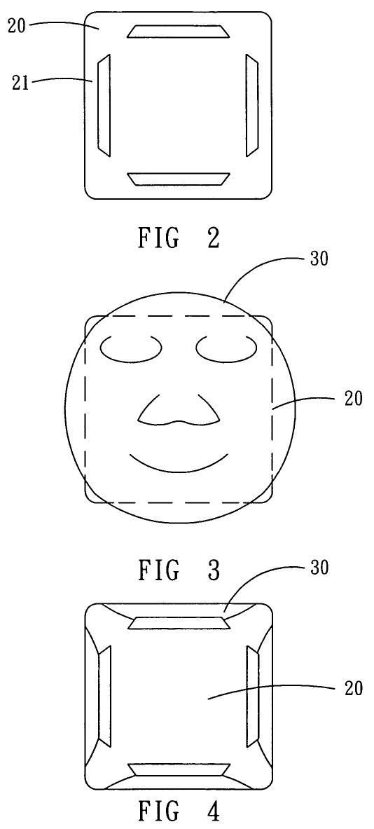

Referring to FIG. 2, the method of the present invention comprises the steps of

(1) cutting openings of certain sizes at certain positions into a separating foil;
(2) soaking a beauty mask body in a liquid beauty agent;
(3) placing the beauty mask on the separating foil, tucking jutting out parts thereof into the openings;
(4) packing the beauty mask and the separating foil in a bag;
(5) sealing the bag; and
(6) freezing the bag at a temperature of approximately −20° C. for storage.

By storing beauty masks at freezing temperatures, effectivity and freshness thereof are maintained, quality is not impaired, and unlimited storage is possible, without usage of antiseptic agents and antioxidants.

The beauty mask of the present invention is during summer after storage at freezing temperatures directly applicable to the skin, providing a pleasant cool and fresh feeling. Should the beauty mask feel too cold, storage at room temperature for a couple of minutes before applying is preferred. If applied at cool temperatures, the beauty mask of the present invention tightens the skin effectively and allows for relativiely good absorption of agents. Using alcohol, as in conventional beauty masks, leads to a fresh feeling, but is harmful to the skin.

For transporting and storing the beauty mask of the present invention, insolating boxes, e.g., styrofoam boxes, are preferably used.

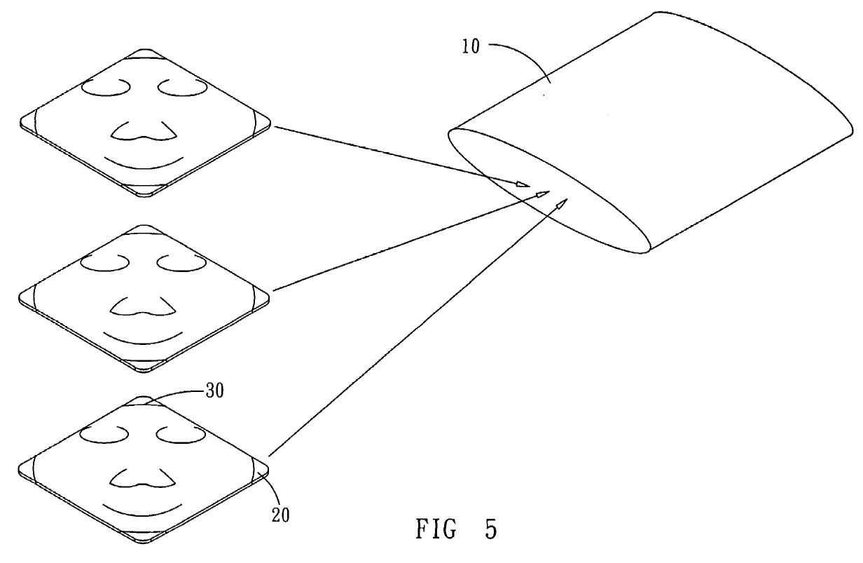

Referring to FIGS. 2-5, the beauty mask storing and dispensing device of the present invention comprises a bag 10, a plurality of separating foils 20, and a plurality of beauty masks 30. The plurality of separating foils 20 and the plurality of beauty masks 30 are placed in the bag 10.

The bag 10 provides space for accommodating the plurality of separating foils 20 and the plurality of beauty masks 30.

Each of the plurality of separating foils 20 is shaped like a rectangle, made of environmentally friendly material, e.g., PET, which is light and heat-resistant, and has several openings 21 for fastening one of the plurality of beauty masks 30.

Each of the plurality of beauty masks 30 is laid on one of the plurality of separating foils 20, with parts that jut out bent rearward and tucked into the openings 21 thereof.

Thereby, the plurality of beauty masks 30 are respectively fastened to the plurality of separating foils 20 and, when stacked on each other and placed in the bag 10, thereby separated from each other and prevented from sticking to each other. Dispensing single beauty masks 30 is readily done, without any need to separate single beauty masks 30 from each other, thus avoiding a risk of contamination thereof.

In contrast thereto, conventional beauty masks are usually folded and the packed to reduce volume, so that conventional beauty masks are not easily dispensed and a risk of contamination thereof is increased.

While a preferred embodiment of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

1. A beauty mask, comprising a mask body and a liquid beauty agent, said liquid beauty agent further comprising:
coconut milk to a fraction of 70%-99%,
royal jelly to a fraction of 0.1%-10%,
vegetable enzymes to a fraction of 0.1%-5%,
aloe to a fraction of 0.1%-5%, and
loofah to a fraction of 0.1%-5%,
forming a liquid beauty agent, keeping the beauty agent at a temperature of 5° C. to 10° C.,
wherein said mask body is soaked in said liquid beauty agent.

2. The beauty mask of claim 1, wherein said mask body is made of nonwoven fabric.

3. The beauty mask of claim 1, wherein said vegetable enzymes are papayase.

4. A method for storing beauty masks, comprising the steps of
(1) cutting openings of certain sizes at certain positions into a separating foil;
(2) soaking a beauty mask body in a liquid beauty agent;
(3) fastening said beauty mask on said separating foil;
(4) packing said beauty mask on said separating foil in a bag;
(5) sealing said bag; and
(6) freezing said bag for storage.

5. The method of claim 4, wherein said separating foil is of rectangular shape, having four edges, with said openings cut along said four edges, and said beauty mask is fastened on said separating foil by tucking jutting out parts thereof into said openings.

6. The method of claim 4, wherein said bag is stored at a temperature of approximately −20° C.

7. A beauty mask storing and dispensing device, comprising
a bag;
at least one separating foil; and
at least one beauty mask, fastened to said at least one separating foil by tucking jutting out parts thereof into openings thereof;
wherein stacking said at least one beauty mask on each other will not result in said at least one beauty mask sticking together, so that dispensing thereof is easy.

8. The beauty mask storing and dispensing device, of claim 7, wherein said bag is made of PET.
